(19) United States
(12) Patent Application Publication
(10) Pub. No.: US 2024/0350760 A1
(43) Pub. Date: Oct. 24, 2024

(54) PNEUMATIC SYSTEM FOR AN ANAESTHESIA SYSTEM

(71) Applicant: Drägerwerk AG & Co. KGaA, Lübeck (DE)

(72) Inventors: Andreas BRANDT, Lübeck (DE); Peter BACH, Lübeck (DE); Dennis STURM, Lübeck (DE)

(21) Appl. No.: 18/687,364

(22) PCT Filed: Aug. 5, 2022
(86) PCT No.: PCT/DE2022/100565

§ 371 Date: Feb. 28, 2024

(30) Foreign Application Priority Data
Sep. 1, 2021 (DE) 10 2021 122 598.1

(51) Int. Cl. A61M 16/00; A61M 16/08; A61M 16/10; A61M 16/20

(52) U.S. Cl. CPC A61M 16/024; A61M 16/0003; A61M 16/0063; A61M 16/0078; A61M 16/0093; A61M 16/0891; A61M 16/1015; A61M 16/202; A61M 16/208; A61M 2016/0027; A61M 2016/0039; A61M 2205/071; A61M 2205/3327; A61M 2205/3334; A61M 2205/3344; A61M 2205/50; A61M 2205/75; A61M 2230/40

(57) ABSTRACT

A pneumatic system (55) for an anesthesia system, includes an internal closed-circuit system (34) and with an external closed-circuit system (54). The internal closed-circuit system (34) has a flush valve assembly (49). The flush valve assembly (49) can be brought into an open state by a control unit (200) on the basis of a current tidal volume.

### CROSS REFERENCE TO RELATED APPLICATIONS

This application is a United States National Phase Application of International Application PCT/DE2022/100565, filed Aug. 5, 2022, and claims the benefit of priority under 35 U.S.C. § 119 of German Application 10 2021 122 598.1, filed Sep. 1, 2021, the entire contents of which are incorporated herein by reference.

### TECHNICAL FIELD

The present invention pertains to an assembly of components into a pneumatic system for an anesthesia device. Anesthesia devices are used for safely carrying out a general anesthesia. Modern anesthesia devices have a closed breathing system, in which the majority of the breathing gas does not leave the device. These are also called closed anesthesia systems. The exhaled carbon dioxide is absorbed by breathing lime and only the portion of gas (e.g., oxygen) that has been used up is fed into the closed circuit in the form of fresh gas. This process has the advantage that the substances (general anesthetics) used for the anesthesia can be utilized efficiently.

### BACKGROUND

Different variants of anesthesia devices with a radial compressor (blower) are described in U.S. Pat. No. 5,875,783 A. FIG. 6 of U.S. Pat. No. 5,875,783 A as well as the parallel property right DE 19714644 C2 show a pneumatic system, which can be equipped with a radial compressor. Compared to piston drives, radial compressors offer the advantage that, especially when stopped (n=0) or at very low or low speeds of rotation of the impeller in the radial compressor, both flows of quantities of gas with an operating speed of rotation of the impeller and flows of quantities of gas against the operating direction of rotation of the impeller are possible. Quantities of gas are delivered in the operating direction of rotation towards a patient. This offers many advantages for the configuration of anesthesia devices or ventilators, as it is also described in U.S. Pat. No. 5,875,783 A. Thus, the patient can also exhale against the operating direction of rotation through the radial compressor against a low flow resistance. Based on the description of FIG. 6 of DE 19714644 C2, the functions of the pneumatic system will be described for explaining the function and the advantages of the pneumatic system according to the state of the art, as it is described in DE 19714644 C2.

A radial compressor draws in an anesthetic gas, formulated as a mixture of oxygen or air with nitrous oxide and evaporated anesthetic from a so-called fresh gas line and, in addition, buffered breathing gas from a manual breathing bag as the gas to be inhaled during the inhalation. If the pressure level in the lungs of the patient is lower than the pressure level at the radial compressor, this inhalation gas then reaches the patient and enters into the patient via a carbon dioxide absorber and through an inspiratory nonreturn valve via ventilation hoses, a patient connection element (patient Y-piece) and an airway access (breathing mask, endotracheal tube, tracheostomy). As soon as the pressure conditions reverse, i.e., as soon as a pressure level in the lungs of the patient is above the pressure level at the radial compressor, the gas flows from the patient through an expiratory nonreturn valve and through the radial compressor into the manual breathing bag. The pressure level at the outlet of the radial compressor depends on the quantity of gas being delivered, which can be set by varying the speed of rotation, and on the flow resistances in the pneumatic system. A pressure relief in the circuit can be brought about via an adjustable resistance into an anesthetic gas scavenging line (purging line). A position shown by broken lines must be selected for the adjustable resistance for an automatic ventilation. As an alternative, the valve may also be bridged over pneumatically in another manner with a bypass valve (ABV). The closed circuit is driven during the manual ventilation by the manual breathing bag, and a valve (APL valve—adjustable pressure-limiting valve) limits the airway pressure. Manual ventilation at an elevated pressure level is possible with the radial compressor running. The quantity of inhaled gas is measured with a flow sensor and the measured data can be used in an electronic control unit with drive motor for controlling the ventilation. It is made possible with this pneumatic system that the patient can inhale and exhale spontaneously at each pressure level.

In certain operating situations of an anesthesia device, especially in operating situations in which only small quantities of fresh gas are introduced into the pneumatic system during largely stable phases of the anesthesia being carried out, i.e., a majority of the anesthetic gas circulates in a closed system to and fro in the pneumatic system between the flow sensor and the patient, the situation arises that an oscillating volume may become established in the pneumatic system. The effect occurs that the patient receives only small quantities of fresh oxygen or that the oxygen being fed as fresh oxygen enters at least partially—or in some situations even predominantly-into the anesthetic gas scavenging system (AGSS: anesthesia gas scavenging system), and the patient largely rebreathes his own, previously exhaled gas, without a sufficient removal of carbon dioxide by the carbon dioxide absorber taking place.

It should, in addition, be noted in view to the volatile anesthetic gases being used, that saving of anesthetic gases during the performance of so-called low-flow anesthesia with small quantities of fresh gas flow in closed or partially open anesthesia systems entails significant cost savings, on the one hand, and, in addition, a reduction of the release of anesthetic gases into the surrounding area is nevertheless achieved when an avoidance of needless consumption of anesthetic is guaranteed by the circulation. The reduction of quantities of anesthetic gases released into the surrounding area is also very welcome for reasons of climate protection, because volatile anesthetic gases, such as desflurane, isoflurane, enflurane, sevoflurane, halothane, act as gases harmful for the atmosphere, similarly to hydrocarbons or methane, because these gases contribute to the warming of the surface of the earth by additionally absorbing infrared radiation.

The situation with the formation of such an oscillating volume occurs during the operation especially when the tidal volume V_{T}, which is fed to the patient during the inhalation by the activation of the radial compressor and which flows back again into the manual breathing bag from the patient through the pneumatic system during the exhalation due to the pressure reduction by means of a temporary or partial deactivation or a reduction of the speed of rotation of the radial compressor, has an order of magnitude that is equal to the volume of the pneumatic system. The essential part of the oscillating volume in the pneumatic system is formed now between the location of the feed of the fresh gas and the location of the branching of the pneumatic system into an inspiratory path and an expiratory path.

The volume within the pneumatic system is determined mainly by the volume of the carbon dioxide absorber and by the construction of the pneumatic components. Since the oscillating volume is influenced predominantly by the construction of the pneumatic components and by the manner of integration of the pneumatic components within the ventilator and/or anesthesia device and since, moreover, the volume of the carbon dioxide absorber cannot be reduced without appreciable drawbacks in respect to reduced durations of use of surgical interventions, there is a need for providing a solution which provides the advantages of a cost-effective pneumatic system with a radial compressor, namely, that the advantages of the cost-effective pneumatic system with a radial compressor can also be fully exploited during an operation with small tidal volumes.

An alternative solution to the problem in respect to the oscillating volume could be achieved by avoiding situations with small tidal volumes. This would be possible, in principle, by limiting the tidal volumes that can be set to a lower value of a minimal tidal volume in the anesthesia device. Use in certain patient groups, especially infants, toddlers and younger children, would thus no longer be possible with such an anesthesia device. Such a limitation to adult patients does not represent an adequate approach to solving the problem described.

### SUMMARY

Based on the state of the art, an object is thus to provide a device and a process for dispensing different tidal volumes for an anesthesia system. In particular, reliable dispensing of small tidal volumes by the anesthesia system shall be made possible by the device and process.

The object is accomplished by features according to the invention.

The object is accomplished by an assembly for a pneumatic system having features according to the invention.

The object is accomplished, furthermore, by a process for operating the pneumatic system with features according to the invention.

The process for operating the pneumatic system may also be configured as a computer program, as a part of a computer program, as a computer program product or as a part of a computer program product having features according to the invention.

The present invention will be explained in more detail on the basis of the following description partly with reference to the figures.

Embodiments offer possibilities of a pneumatic system for use as a part of an anesthesia system.

Embodiments create, in addition, possibilities for configuring a process for operating a pneumatic system as a part of an anesthesia system.

Further features and details of the present invention and advantageous embodiments appear from this disclosure including the description and from the drawings.

Features and details that are described in connection with the pneumatic system according to the present invention also apply, of course, in connection with the process or computer program according to the present invention and also vice versa, so that reference is or can always mutually be made to the individual aspects of the present invention concerning the disclosure. References used in this connection refer to the further embodiment of the subject of the principal claim by the features of the respective subclaim and shall not be understood to represent an abandonment of the inclusion of an independent, concrete protection for the combination of the features of the referred subclaims. Furthermore, it shall be assumed in respect to an interpretation of the claims as well as of the description in case of a more specific concretization of a feature in a dependent claim that such a limitation is not present in the respective preceding claims as well as in a more general embodiment of the concrete device or process. Any reference in the description to aspects of dependent claims shall accordingly also expressly imply a description of optional features even without a special reference. Finally, it should be pointed out that the pneumatic system being proposed here may also be improved corresponding to the process claims and conversely, for example, by the pneumatic system comprising components and/or devices that are intended and/or configured for carrying out one or more process steps or by the process comprising steps that can be carried out by means of the pneumatic system or are suitable for the operation of the pneumatic system. Thus, features and details that are described in connection with the pneumatic system being proposed are, of course, also valid in connection with and in respect to a process carried out during the operation of the pneumatic system and also vice versa, so that reference is and can always mutually be made to the individual aspects of the present invention concerning the disclosure.

According to a first aspect of the present invention, embodiments are shown, which show assemblies of components into a pneumatic system for an anesthesia system,
wherein the assembly has at least the following components:
a control unit,
a radial compressor.
an internal closed-circuit system with:
   a carbon dioxide absorber,
   a ventilation system connection element (internal Y-piece),
   an inspiratory path with an inspiratory nonreturn valve,
   an expiratory path with an expiratory nonreturn valve,
   a flush valve assembly (purging valve),
an APL valve assembly.
a breathing bag,
a mixing unit,
a first pressure sensor P1, and
a first flow sensor V1.

The assemblies for the anesthesia system is complemented during the operation of the anesthesia system by an external closed-circuit system having the following components:
with a patient connection element (patient Y-piece),
with an inspiratory ventilation hose, and
with an expiratory ventilation hose.

The external closed-circuit system is used for the pneumatic and fluidic connection of the patient to the anesthesia system. The ventilation hoses are connected for this with inspiratory and expiratory ports, which are usually configured each as a cone, on the device side, and are connected to the patient connection element on the patient side. An element for feeding gas into the patient, for example, an endotracheal tube, a nasal mask or a tracheostomy (access to the trachea) is connected to the patient connection element.

The first flow sensor V1 is intended for the measurement-based detection and/or determination of measured signals, which indicate quantities of gas and flow directions of quantities of gas in the internal closed-circuit system. The first flow sensor V1 provides these measured signals to the control unit.

The first pressure sensor P1 is intended for a measurement-based detection and/or determination of measured signals, which indicate a pressure level in the internal closed-circuit system. The first pressure sensor P1 provides these measured signals to the control unit.

The breathing bag represents a reservoir in the internal closed-circuit system, which takes up the quantities of breathing gas exhaled by the patient.

The flush valve assembly provides a controllable dispensing valve including the pneumatic and electrical connection elements and connections necessary for the operation, as well as for the operation of the signal lines or data lines. The control unit is configured to control the flush valve by means of the signal or data lines, i.e., especially to bring about an open state or a closed state of the dispensing valve.

The APL valve assembly provides an adjustable pressure-limiting valve (APL valve) in the pneumatic system including the pneumatic and electrical connection elements and connections necessary for the operation, APL denoting “adjustable pressure limiting.” The pneumatic system makes possible with the mixing unit a mixing of gases to form a gas mixture, which is suitable and is intended for carrying out an anesthesia and can be made available to a patient by the pneumatic system. The gas mixture as a so-called “fresh gas” (FG) consists, in addition to oxygen, of air and/or nitrous oxide and usually at least one volatile anesthetic (halothane, desflurane, enflurane, sevoflurane, isoflurane). The radial compressor is configured and intended to deliver the gas mixture to the patient. The delivery to the patient takes place in the internal closed-circuit system via the inspiratory path, in which an inspiratory nonreturn valve is arranged, which prevents gases from flowing back from the patient into the inspiratory path. The backflow from the patient takes place via the expiratory path and the breathing system connection element (internal Y-piece) into the breathing bag. An expiratory nonreturn valve, which prevents gases from flowing back to the patient, is arranged in the expiratory path. The feed of gas to the patient takes place by means of the patient connection element, at which the inspiratory path is merged with and connected to an inspiratory ventilation hose and the expiratory path is merged with and connected to an expiratory ventilation hose. During automatic ventilation, the radial compressor delivers a breathing gas mixture from the mixing unit and from the breathing bag as inhalation gas during the phase of inhalation. This inhalation gas passes to and into the patient via the inspiratory path through the carbon dioxide absorber, through the inspiratory nonreturn valve via the external closed-circuit system with the ventilation hoses and the patient connection element (patient Y-piece) and an airway access (breathing mask, endotracheal tube, tracheostomy). During automatic ventilation, the exhaled gas flows into the breathing bag during the phase of exhalation from the patient through the expiratory nonreturn valve and through the radial compressor.

The APL valve assembly is switched during automatic ventilation such that no substantial quantities of exhaled gas can flow from the pneumatic system into the anesthetic gas scavenging line (purging line).

The external closed-circuit system is used to feed fresh breathing gases to the patient and to remove consumed breathing gases from the patient into the internal closed-circuit system.

The control unit is configured and intended to organize, control, i.e., control or regulate an operation and/or a process of the pneumatic system and/or of the anesthesia system. The control unit is preferably formed from components (μC, μP, PC) with corresponding operating system (OS), memory (RAM, ROM, EEPROM), as well as SW code, software for sequence control, control, i.e., control and regulation. In at least some exemplary embodiments, further electronic elements, for example, components for signal detection (ADμC), signal amplification, for analog and/or digital signal processing (PLD, ASIC, FPGA), components for analog and/or digital signal filtering (PLD, DSP, FPGA, GAL, μC, μP), signal conversion A/D converter) are assigned or connected to the control unit.

The control unit controls the operation of the pneumatic system in the anesthesia system for carrying out an anesthesia or for performing inhalation anesthesia with provision of mechanical ventilation with the addition of anesthetic gases, wherein measured signals of the first pressure sensor P1 are taken into consideration by the control unit for controlling inspiratory and expiratory pressure levels over the time course of inhalation and exhalation. The control unit can determine the breathing phases with the sequence of phases of inhalation and phases of exhalation even during spontaneous breathing of the patient on the basis of the measured signals of the first pressure sensor P1, and at times also on the basis of the measured signals of the first flow sensor V1. The control unit can control, i.e., set, control or regulate the quantities of breathing gas fed to the patient and the sequence of inhalation and exhalation, the ventilation mode based on variations of the speed of rotation of the radial compressor, taking into account the measured signals of the first flow sensor V1 and the measured signals of the first pressure sensor P1. During the operation of the pneumatic system, the control unit continuously carries out a measured signal detection of the first pressure signal P1 and of the first flow sensor V1 with a subsequent measured signal analysis, and the current inspiratory tidal volume V_{T} is calculated now during the phase of inhalation on the basis of the measured signals of the first flow sensor V1 and it is compared to a lower threshold value V_{T_Limit_1} or to an upper threshold value V_{T_Limit_2}. The control unit is configured to control, i.e., to set, to control or to regulate the flush valve assembly on the basis of the comparison of the current tidal volume (V_{T}) to the threshold values V_{T_Limit_1}, V_{T_Limit 2}, especially in order to switch the flush valve assembly between a closed state and an open state.

In case the current tidal volume V_{T} undershoots the lower threshold value V_{T_Limit_1}, the flush valve assembly is brought into an open state. An operating state becomes established, in which quantities of exhaled gases from the internal closed-circuit system can flow out of the pneumatic system through the flush valve assembly into the anesthetic gas scavenging system.

In case the current tidal volume V_{T} exceeds the current tidal volume V_{T}, the flush valve assembly is brought into a closed state. An operating state becomes established, in which no quantities of exhaled gases can flow from the internal closed-circuit system through the flush valve assembly and into the anesthetic gas scavenging system from the pneumatic system.

The range of the lower threshold value V_{T_Limit_1} is selected now such that it is ensured during the operation that no quantities of breathing gases exhaled by the patient can flow to and fro as a kind of oscillating volume between the breathing bag and the breathing system connection element (internal Y-piece).

The range of the upper threshold value V_{T_Limit_2} is selected now such that in case of a tidal volume, which is applied into the patient and which is detected by measurement by means of the first flow sensor V1, and which tidal volume is markedly above the volume of the internal closed-circuit system and of the carbon dioxide absorber, the quantities of breathing gases exhaled by the patient can flow possibly even several times to and fro between the breathing bag and the breathing system connection element (internal Y-piece), without the flush valve assembly being brought into the open state, and an outflow into the anesthetic gas scavenging system is thus possible.

The lower threshold value V_{T_Limit_1} may correspond in an advantageous embodiment to twice the oscillating volume between the breathing bag and the breathing system connection element (internal Y-piece).

A range for a lower threshold value V_{T_Limit_1} below 500 mL can be mentioned as an example.

A range for an upper threshold value V_{T_Limit 2} above about 750 mL to 1,000 mL can be mentioned as an example.

In an advantageous dimensioning, the upper threshold value V_{T_Limit_2} can correspond to twice the value of the lower threshold value V_{T_Limit_1}.

In a preferred embodiment of the anesthesia system with the pneumatic system, a first pressure sensor P1 may be arranged for detecting a pressure level occurring in the closed-circuit system. The first pressure sensor P1 is configured for detecting and providing measured signals, which indicate a pressure level occurring in the internal closed-circuit system, for the control unit.

In a preferred embodiment of the anesthesia system with the pneumatic system, an additional pressure sensor P2 may be arranged for detecting a pressure level in the pneumatic system. The additional pressure sensor P2 is intended for a measurement-based detection and/or determination of measured signals, which indicate a pressure level at the flush valve assembly. The additional pressure sensor P2 is configured to provide these measured signals to the control unit. The control unit is configured to also include the measured signals, which indicate the pressure level occurring in the internal closed-circuit system, when the state of change of the flush valve assembly is brought about.

In a preferred embodiment of the anesthesia system with the pneumatic system, an additional flow sensor V2 may be arranged in the expiratory path as an expiratory flow sensor for detecting exhaled quantities of breathing gases. The additional flow sensor V2 is intended for a measurement-based detection and/or determination of measured signals, which indicate quantities of gas exhaled by the patient in the expiratory path. The additional flow sensor V2 provides these measured signals to the control unit. The control unit is configured to also include the measured signals, which indicate quantities of flow flowing to or from the patient, when the state of change of the flush valve assembly is being brought about.

In a preferred embodiment of the anesthesia system with the pneumatic system, an oxygen sensor may be arranged for detecting an oxygen concentration of quantities of breathing gases in the internal closed-circuit system and/or in the inspiratory path or expiratory path. The control unit is configured to also include the measured signals, which indicate the oxygen concentration, when the state of change of the flush valve assembly is being brought about. A situation in which an O₂ flush situation is present can be assumed in case of a rapid or abrupt increase in the oxygen concentration to a concentration value of nearly 100%. An activation of the flush valve assembly can thus be brought about at the same time by the control unit into an open state in order to accelerate the gas exchange to the patient.

In a preferred embodiment of the anesthesia system with the pneumatic system, the control unit is configured to also take into consideration during the control of the operation measured signals
e. of the first pressure P1,
f. and/or of the first flow sensor,
g. and/or of the additional pressure sensor P2,
h. and/or of the additional flow sensor V2
i. and/or of the oxygen sensor during the control of the operation when changes of state
a. of the flush valve assembly,
b. of the APL valve assembly,
c. of the radial compressor, and
d. of the fresh gas mixing unit are brought about.

This leads to the advantage that the control unit is able to also take into account the current system behavior and/or the current system state of the anesthesia system, which arise on the basis of changes in the operating and ambient conditions, on the basis of changes of settings on the anesthesia system by the user, on the basis of user interactions with the patient, on the basis of activities of the patient or on the basis of alarm situations in the operation of the anesthesia or ventilation, as well as the state of the flush valve assembly adapted to current situations during the operation within the framework of a holistic control concept.

The control unit is configured in a preferred embodiment of the anesthesia system with the pneumatic system to perform an activation of the flush valve assembly into an open state along with an activation of another valve, especially of an O₂ flush valve. An opening of the flush valve assembly can be made possible in this manner simultaneously with the activation of the so-called O₂ flush valve. Such an O₂ flush valve is activated by the user, for example, by means of a button element or switching element, and it is used to rapidly supply or flood the pneumatic system with a high concentration of oxygen (O₂). The O₂ flush valve is usually arranged in the pneumatic system such that quantities of gas ranging from 30 L/min to 50 L/min with an oxygen concentration of 100% are sent directly to the patient, usually also bypassing the mixture preparation unit and/or the anesthetic dispensing unit. This supply/flooding of the pneumatic system with a high concentration of oxygen can be supported by simultaneously opening the flush valve assembly. As a result, the time elapsing until a high concentration of oxygen is reached in the pneumatic system after the activation of the O₂ flush valve can advantageously be reduced further. In one technical configuration of this preferred embodiment, the control unit may be configured, for example, to detect or read back a state of the button element or switching element or the state of the O₂ flush valve, e.g., with a switching contact. Based on this, the control unit can then initiate an opening of the flush valve assembly.

In a preferred embodiment of the anesthesia system with the pneumatic system, the flush valve assembly may be configured with an additional functionality as a pressure relief valve. The functionality of the pressure relief valve may be configured as an electromechanical valve controllable by the control unit. The opening of the valve for bringing about a pressure relief above a predefined pressure level into the anesthetic gas scavenging system by the control unit can take place in this embodiment of an electromechanical pressure relief valve on the basis of the measured signals of the first pressure sensor P1 and/or of the additional pressure sensor P2. The control unit carries out a comparison of the measured signals of the additional pressure sensor P2 with a lower threshold value P_{Limit_2} in order to bring about and control a state of an opening for pressure relief into the anesthetic gas scavenging system at the flush valve assembly when the lower threshold value P_{Limit_2} is exceeded by the current measured signals of the additional pressure sensor P2. A functionality as a pressure relief valve may be configured in an alternative embodiment as a mechanical valve that can be set at a variable or fixed pressure level by means of spring loading.

A process according to the present invention for operating an anesthesia system will be described below according to another aspect of the present invention. The process makes possible a safe operation of an anesthesia system even for small tidal volumes. The control unit—or another unit configured in a suitable manner for carrying out process steps-carries out during the operation of an anesthesia system with a pneumatic system with at least the following components:
radial compressor, carbon dioxide absorber, gas supply to a patient, a flush valve assembly for determining operating states a process with the following sequence of steps:
   a. measured value acquisition with detection of measured signals of a first pressure sensor P1 and of a first flow sensor V1,
   b. measured value analysis for determining an operating state of the anesthesia system on the basis of the measured signals, and
   c. adaptation of an operation of the anesthesia system takes place depending on the determined operating state.

It is determined during the determination of the operating states whether an operating state in which certain partial quantities or quantities of carbon dioxide-containing and oxygen-depleted exhaled gases exhaled by the patient flow back to the patient.

The process for operating an anesthesia system is configured in the following manner in a preferred embodiment for configuring the process steps:
a. Measured value acquisition
   detection of a measured signal of the first pressure sensor P1,
   detection of a measured signal of the first flow sensor V1
b. Measured value analysis for determining an operating state
   determination of an operating state on the basis of the measured signals of the first pressure sensor and of the first flow sensor, whether an operating state is given, in which certain partial quantities or quantities of carbon dioxide-containing and oxygen-depleted exhaled gases exhaled by the patient flow back to the patient and a situation with pendulum breathing thus occurs such that the current inspiratory tidal volume V_{T} is calculated during the phase of inhalation on the basis of the measured signals of the first flow sensor V1 and the current tidal volume V_{T} is compared to a lower threshold value V_{TLimit}, especially to a lower threshold value V_{TLimit_1}. The measured value analysis provides on the basis of the measured signals of the first pressure sensor P1 the phase of breathing with the phases of inhalation and phases of exhalation taking place.
c. Adaptation of the operation
   when the current tidal volume V_{T} undershoots the threshold value V_{TLimit_1}, especially the lower threshold value V_{TLimit_1}, the flush valve assembly is activated into an open state. This leads to a situation in which the pneumatic system is operated as an open anesthesia system with open circuit, with feed of quantities of fresh gas and scavenging of exhaled quantities of breathing gas. Such an operation as an open anesthesia system reliably prevents the patient from being able to inhale certain partial quantities or quantities of a carbon dioxide-containing exhaled gas. No pendulum breathing can occur for the patient during the operation as an operation as an open anesthesia system.

An integration of these process steps into an operation of an anesthesia system makes automatically possible a safe ventilation for the patient with small tidal volumes. Integration of these process steps into an operation of an anesthesia system makes possible a ventilation of patients both during the operation as a closed anesthesia system and during operation as an open anesthesia system.

In a preferred embodiment of the process, the activation of the flush valve assembly may not take place into the open state, for example, also depending on currently used ventilation parameters, in each phase of breathing, but only temporarily or proportionally, so that the flush valve assembly or the flush valve SV is also activated for opening, e.g., especially only in every other or every third phase of breathing. Such an embodiment offers the advantage that a state with a continual switching between closed and open anesthesia system can be avoided. Such states may occur especially as undershooting when the current tidal volume V_{T} and the lower threshold value V_{TLimit_1} have only slight differences. Breathing phases may be defined in the sense of the present invention both as a phase of inhalation and as a phase of exhalation. A phase of inhalation with a subsequent phase of exhalation shall also be defined as a breathing phase. A phase of exhalation with a subsequent phase of inhalation shall, moreover, also be understood to fall under the term breathing phase.

In a preferred embodiment of the process, the transition from a closed anesthesia system to an open anesthesia system can be brought about by means of an activation of the flush valve assembly via a first transition range with a defined volume range of tidal volumes. In a preferred embodiment of the process, the transition from an open anesthesia system to a closed anesthesia system can take place by means of a deactivation of the flush valve via a second transition range with a defined volume range of tidal volumes. The transition in the first transition range from the closed anesthesia system to the open anesthesia system can take place continuously, slidingly or stepwise in a plurality of steps. The transition in the second transition range from the open anesthesia system to the closed anesthesia system can take place continuously, slidingly or stepwise in a plurality of steps. The ranges of the tidal volumes of the first transition range and of the second transition range may be configured as ranges of tidal volumes that are different from one another.

The ranges of the tidal volumes of the first transition range and of the second transition range may be configured as mutually identical ranges of tidal volumes. A situation which can be called “half open anesthesia system” or “partially open anesthesia system” arises during the operation of the anesthesia system in the transition ranges between a “closed anesthesia system” and an “open anesthesia system.” The flush valve SV of the flush valve assembly is permanently closed during the phases of exhalation in a “closed anesthesia system.” The flush valve SV of the flush valve assembly is permanently open during the phases of exhalation in an “open anesthesia system.” The flush valve SV of the flush valve assembly is either permanently open or permanently closed in a “partially open anesthesia system” or “partially closed anesthesia system,” and the flush valve SV is rather open only for a part of the exhalation time.

In a preferred embodiment of the process, an alternation between a closed anesthesia system and an open anesthesia system can be controlled on the basis of information concerning an expiratory volume. This can be implemented technically, for example, such that corresponding information concerning the expiratory volume is available to the control unit or a corresponding measured signal of an expiratory flow sensor V2, which is arranged in or at the anesthesia system, is available to the control unit.

In a preferred embodiment of the process, an alternation between a closed anesthesia system and an open anesthesia system can be controlled on the basis of information concerning an oxygen concentration in the breathing gas. This can be implemented technically, for example, such that corresponding information concerning an oxygen concentration in the breathing gas is available to the control unit or a corresponding measured signal of an oxygen sensor, which is arranged in or at the anesthesia system, is available to the control unit.

In a preferred embodiment of the process, an alternation or a switching between a closed anesthesia system and an open anesthesia system with an activation of the open state of the flush valve assembly can be triggered combined with an activation of an O₂ flush state. An activation of the O₂ flush state takes place usually by a manual input by a user. The oxygen flowing in is sent through the pneumatic system and can escape through the open flush valve SV or the flush valve assembly into the anesthetic gas scavenging system (AGS). The flushing of the pneumatic system with oxygen can thus take place more efficiently. A manual input for the activation of the O₂ flush state can be configured, for example, as an actuation of an operating element (switch, button, touch display, GUI (Graphical User Interface). In an exemplary embodiment variant, the activations of the O₂ flush state, at the same time the O₂ flush state, and of the open state of the flush valve assembly can take place for an operation as an open anesthesia system at the same time with the same input element.

In another exemplary embodiment variant, the situation of an activated O₂ flush state can be determined by means of an oxygen sensor. The open state of the flush valve assembly can thus be activated together with the activated O₂ flush state.

In a preferred embodiment of the process, a switching between a closed anesthesia system and an open anesthesia system can take place with an activation and/or deactivation of the open state of the flush valve assembly by means of a manual input. A manual input for activating and deactivating the open state of the flush valve assembly can be configured, for example, as an actuation of an operating element (switch, button, touch display, GUI) by a user.

Another exemplary embodiment of a process for operating an anesthesia system is a computer program, a part of a computer program, a computer program product or as part of a computer program product with a program code for carrying out a process being described here when the program code is executed on a computer, on a processor or on a programmable hardware component.

The present invention will now be explained in more detail by means of the following figures and the corresponding description of the figures, without limitations of the general inventive idea. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a circuit diagram showing one of different assemblies of a pneumatic system;

FIG. 2 is a circuit diagram showing another of different assemblies of a pneumatic system;

FIG. 3 is a circuit diagram showing another of different assemblies of a pneumatic system;

FIG. 4 is a circuit diagram showing another of different assemblies of a pneumatic system;

FIG. 5 is a circuit diagram showing another of different assemblies of a pneumatic system;

FIG. 6 is a circuit diagram showing another of different assemblies of a pneumatic system;

FIG. 7 is a graph showing operating states of an anesthesia system;

FIG. 8 is a graph showing operating states of an anesthesia system; and

FIG. 9 is a schematic sequence view showing the operation of an anesthesia system.

### DESCRIPTION OF PREFERRED EMBODIMENTS

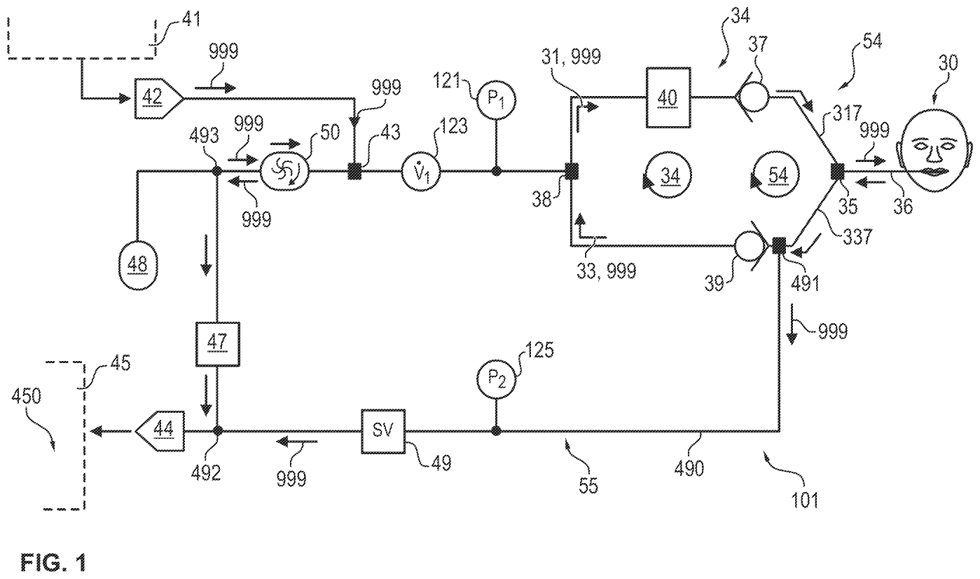

Referring to the drawings, FIGS. 1 through 5 show different embodiments of assemblies 101, 102, 103, 104, 105 of pneumatic systems suitable for anesthesia devices. Identical elements in FIGS. 1 through 5 are designated by the same reference numbers in FIGS. 1 through 5.

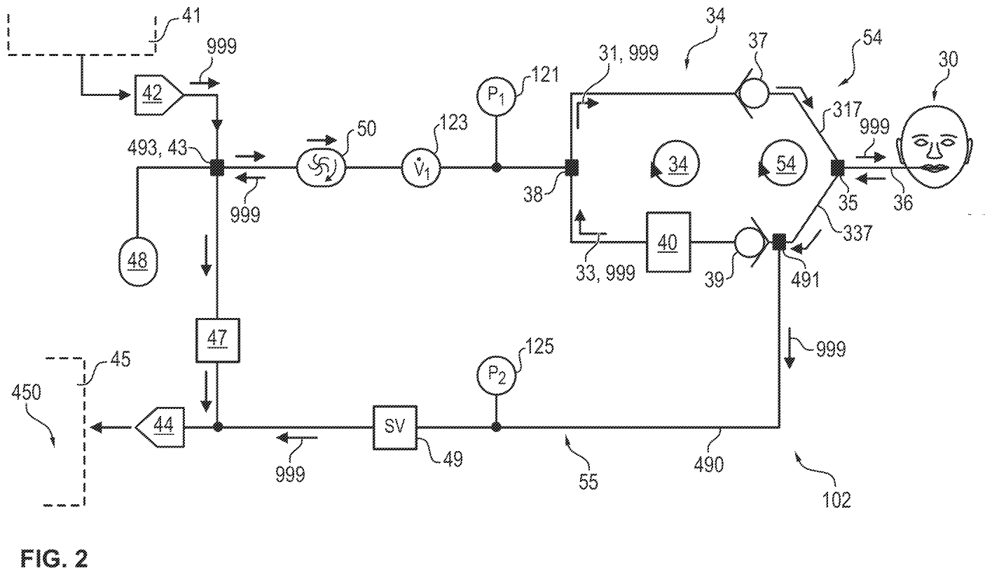

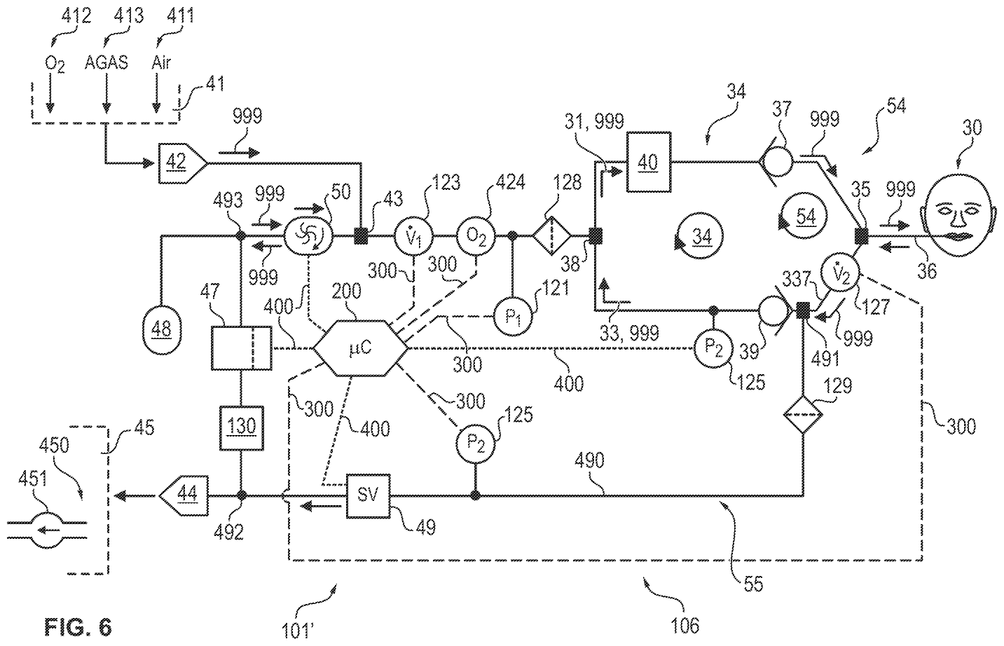

FIG. 6 shows an assembly 106. The assembly 106 is obtained as a variant 101′, supplemented with drawings, of the assembly 101 of FIG. 1. Identical elements in FIGS. 1 and 6 are designated by the same reference numbers as in FIGS. 2, 3, 4, 5 as well. Unlike in FIG. 1, control lines and data lines 300, 400 are also shown and drawn in addition to the gas-carrying connections. The essential functionalities of the assemblies 101, 101′, 102, 103, 104, 105, 106 are explained as examples on the basis of FIG. 1 or FIG. 6 for the assembly 101, 106 and 101′, and the explanations can also be applied to the assemblies 102, 103, 104, 105 of FIGS. 2, 3, 4, 5. The differences are then explained each in detail in respect to the respective peculiarities in the respective figure descriptions for the assemblies 101, 101′, 102, 103, 104, 105, 106.

FIG. 1 and FIG. 6 show the assemblies 101, 101′, 106 of components of a pneumatic system of an anesthesia system with a radial compressor 50 as a breathing gas drive with a carbon dioxide absorber 40, with an inspiratory path 31 and with an expiratory path 33. The inspiratory path 31 and the expiratory path 33 are configured and intended for feeding a breathing gas mixture consisting of breathing gas enriched with anesthetic gases and oxygen via a patient connection element (Y-piece) 35 to a patient 30. Flow arrows 999 show the directions of gas flows within the assembly 101, 101′ (FIG. 6). The carbon dioxide absorber 40 is arranged in the inspiratory path 31 in these assemblies 101, 101′. The supply 42 of fresh gas from a mixing unit 41 into the pneumatic system takes place in this assembly 101 at a fresh gas feed position 43 at the outlet of the radial compressor 50.

The patient connection element (Y-piece) 35, a breathing system connection element (internal Y-piece) 38, an inspiratory nonreturn valve 37, and an expiratory nonreturn valve 39 form, together with the carbon dioxide absorber 40, with the inspiratory path 31 and with the expiratory path 33, an internal closed-circuit system 34, in which quantities of breathing gases, directed and guided in the flow direction, flow through the nonreturn valves 37, 39 into an external closed-circuit system 54, and a gas exchange of partial quantities of the quantities of breathing gas with the patient 30 is thus made possible via an inspiratory ventilation hose 317 and an expiratory ventilation hose 337 via the patient connection element (Y-piece) 35 and an access 36 for gas feed (endotracheal tube, nasal mask, tracheostomy). In addition, a quantity of breathing gas is returned from the patient 30 into the internal closed-circuit system 34 via the access 36 and the connection element (Y-piece) 35. A quantity of carbon dioxide exhaled by the patient 30 is removed continuously from the quantity of breathing gas flowing in a closed-circuit flow by means of the carbon dioxide absorber 40. Fresh quantities of breathing gases are fed to the internal closed-circuit system 34 via the breathing system connection element 38. The quantity of carbon dioxide exhaled by the patient 30 must be replaced essentially by oxygen in order to be able to provide a minimum percentage of oxygen with a volume concentration above 21% for the patient 30.

The flush valve assembly 49 with a controllable, i.e., controllable or regulatable flush valve SV 49 is arranged in a scavenging gas branch (purging branch/flush branch) 490, which leads via an expiratory branch 491 at the expiratory nonreturn valve 39 to a branch 492 of an anesthetic gas scavenging system (AGSS) 44 and to an APL valve assembly 47 with an APL valve 47. Quantities of exhaled gas can thus flow via the scavenging gas branch 490 with the flush valve SV 49 open into the anesthetic gas scavenging system (AGSS) 44 as well for the disposal of anesthetic gas 45 and they can be disposed of. In case of an additionally opened APL valve 47, the quantities of exhaled gas can then reach via the scavenging gas branch 490 the breathing bag 48 or an inlet 493 of the radial compressor 50 and thus-mixed with newly added quantities of oxygen (O₂), which are provided as fresh gas by the mixing unit 41, of air and anesthetic gas (nitrous oxide (N₂O), as well as volatile anesthetic gases, e.g., halothane, desflurane, isoflurane, sevoflurane)—they can again be used further for the ventilation and anesthesia of the patient 30.

The assembly 101′ according to FIG. 6 is based on the assembly 101 and is complemented with some additional components 300, 400, 411, 412, 413, 128, 129, 130, 451 to form the assembly 106. FIG. 6 shows schematically as a detail the supply 42 of fresh gas (FG) from oxygen (O₂) 412, air 411, nitrous oxide 413 and anesthetic gas 413 by the mixing unit 41 in the assembly 106, 101′. In addition, FIG. 6 shows for illustration in addition to FIG. 1 a control unit 200, data lines, signal lines 300 and control lines 400. These views with the control unit 200, with the data lines, with signal lines 300 and with control lines 400 are not shown in FIGS. 1 through 5 for the sake of clarity, but the control unit, data lines, signal lines and control lines are, of course, also present in FIGS. 1 through 5, and FIG. 6 shall thus therefore also expand the technical representation in this respect for FIGS. 1 through 5 as well. Especially in respect to the components 200, 300, 400, the description of FIG. 6 shall also be implied, in the basic sense, for the understanding of FIGS. 1 through 5. Furthermore, FIG. 6 shows, in series with the APL valve 47, an anesthetic gas scavenging valve 130, which may be configured as a passive, e.g., spring- and/or weight-loaded valve 130 or as a controllable, i.e., controllable or regulatable valve 130. A vacuum source for anesthetic gas disposal 45 as a part of an external device 450 or as a part of the hospital infrastructure 450 is shown schematically. Furthermore,

FIG. 6 shows filter elements 128, 129, which may optionally be arranged at the breathing system connection element 38 or in the scavenging gas branch 490, for the protection of the pneumatic system 55, especially as a protection from contamination in general or from contamination with hospital pathogens, e.g., bacteria or viruses. Furthermore, FIG. 6 shows another flow sensor V2 127, which is arranged in the expiratory branch, preferably close to the patient. The additional flow sensor V2 127 makes it possible to balance the quantities of breathing gas exhaled by the patient 30 and can be used together with the inspiratory flow sensor for balancing, for example, in order to identify situations with leaks or leakages.

Furthermore, FIG. 6 shows an oxygen sensor 424, which is arranged at the outlet of the radial compressor 50 in series with the first flow sensor V1 123. The expiratory flow sensor V2 may be arranged in the internal or external closed-circuit system. The oxygen sensor 424 may be used to control the scavenging valve assembly 49 as a function of the oxygen concentration; for example, it can be inferred in case of an abrupt increase in the detected oxygen concentration to nearly 100% that an O₂ flush situation is present, and the flush valve assembly 49 can then be activated into an open state in order to accelerate the gas exchange in the pneumatic system 101, 101′, 106 and, as a consequence of this, also at the patient 30.

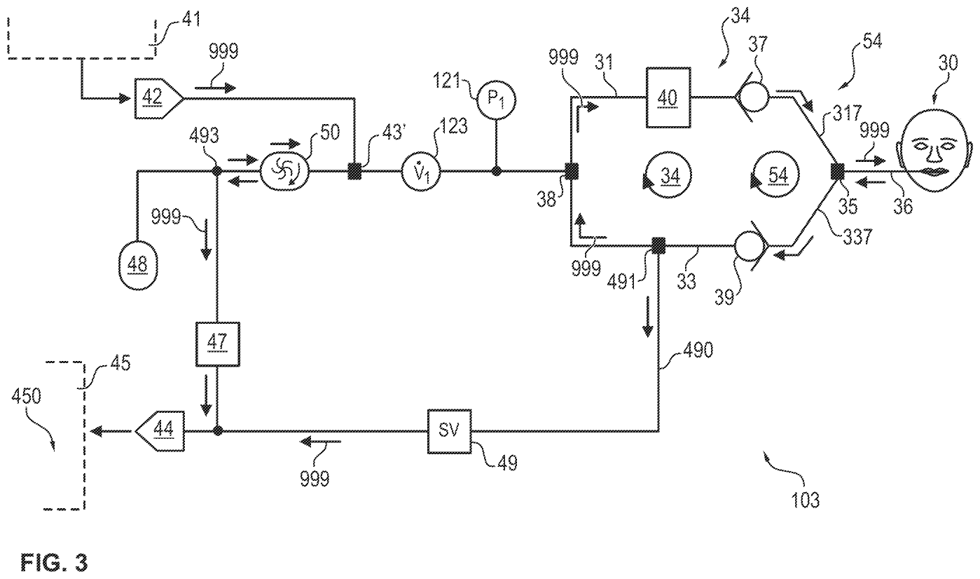

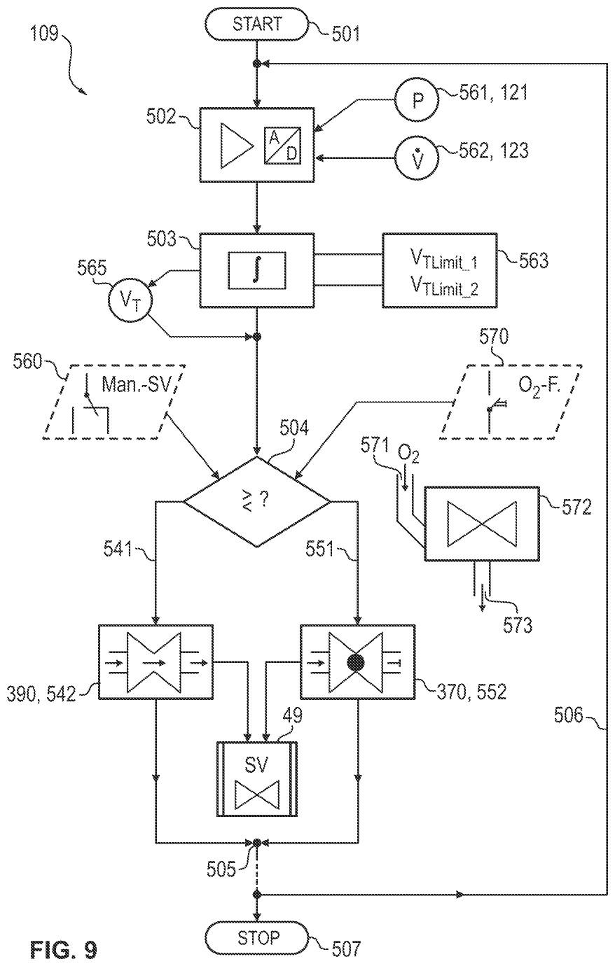

The control unit 200 is configured and intended for organizing, controlling, i.e., controlling or regulating the operation and/or the process of the pneumatic system 101, 101′, 106. The control unit 200 performs continually during the operation of the pneumatic system a measured value acquisition of the first pressure sensor P1 121 and of the first flow sensor V1 123 with a subsequent measured signal analysis, and the current inspiratory tidal volume V_{T} is calculated in the process during the phase of inhalation on the basis of the measured signals of the first flow sensor V1 and it is compared to a lower threshold value V_{T_Limit 1} 563 (FIG. 3) or to an upper threshold value V_{T_Limit 2} 563 (FIG. 9). The control unit is configured to control, i.e., set, control or regulate the flush valve assembly 49 on the basis of the comparison of the current tidal volume V_{T} to the threshold values V_{T_Limit_1} 563 (FIG. 9), V_{T_Limit_2} 563 (FIG. 9) especially in order to switch the flush valve assembly 49 between a closed state 552 (FIG. 9) and an open state 542 (FIG. 9). The flush valve assembly 49 may be configured as a proportional valve or as a two-way valve.

When the current tidal volume V_{T} undershoots one of the threshold values V_{T_Limit_1}, V_{T_Limit_2} 563 (FIG. 9), the flush valve assembly 49 is brought into an open state 542 (FIG. 9). An operating state is obtained, in which quantities of exhaled gases can flow from the internal closed-circuit system 34 through the flush valve assembly 49 into the anesthetic gas scavenging system 44, 45 from the pneumatic system 101, 101′, 106.

When the current tidal volume V_{T} exceeds one of the threshold values V_{T_Limit_1}, V_{T_Limit_2} 563 (FIG. 9), the flush valve assembly 49 is brought into a closed state 552 (FIG. 9). An operating state is obtained, in which no quantities of exhaled gases can flow from the internal closed-circuit system 34 through the flush valve assembly 49 into the anesthetic gas scavenging system 44, 45 from the pneumatic system 101, 101′, 106.

The range of the lower threshold value V_{T_Limit_1} 563 (FIG. 9) is selected now to be such that it is ensured during the operation that no quantities of breathing gases exhaled by the patient 30 can flow to and fro in the manner of an oscillating volume between the breathing system connection element (internal Y-piece) 38 in the internal closed-circuit system 34 and the fresh gas feed 42, 43 or the breathing bag 48.

FIG. 2 shows an alternative embodiment to FIG. 1 with an assembly 102, in which the carbon dioxide absorber 40 is arranged in the expiratory path 33. Flow arrows 999 indicate the directions of gas flows in the assembly 102. The supply 42 of fresh gas (FG) by a mixing unit 41 into the pneumatic system takes place in this assembly 102 at a fresh gas feed position 493 at the inlet 43 of the radial compressor 50. In addition, an additional pressure sensor P2 125 is arranged in this FIG. 2 at the scavenging gas path 490. A balancing of the pressure levels of the pressure sensors P1 121, P2 125, possibly with a comparison to a threshold valve P_{Limit}, also makes it possible to use the flush valve SV 49 in an additional functionality as a pressure relief valve during the operation.

FIG. 3 shows an alternative embodiment to FIG. 1 with an assembly 103, in which the carbon dioxide absorber 40 is arranged in the inspiratory path 31. Flow arrows 999 indicate the directions of gas flows in the assembly 103. The supply 42 of fresh gas (FG) by the mixing unit 41 into the pneumatic system takes place in this assembly 103 at a fresh gas feed position 43′ at the outlet of the radial compressor 50.

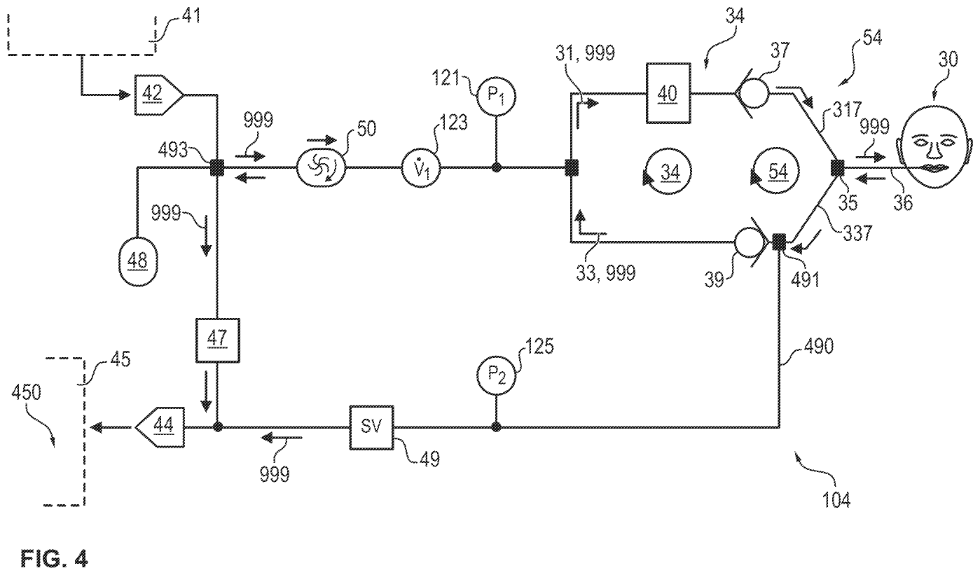

FIG. 4 shows an alternative embodiment to FIG. 3 with an assembly 104, in which the carbon dioxide absorber 40 is arranged in the inspiratory path 31. Flow arrows 999 indicate the directions of gas flows in the assembly 104.

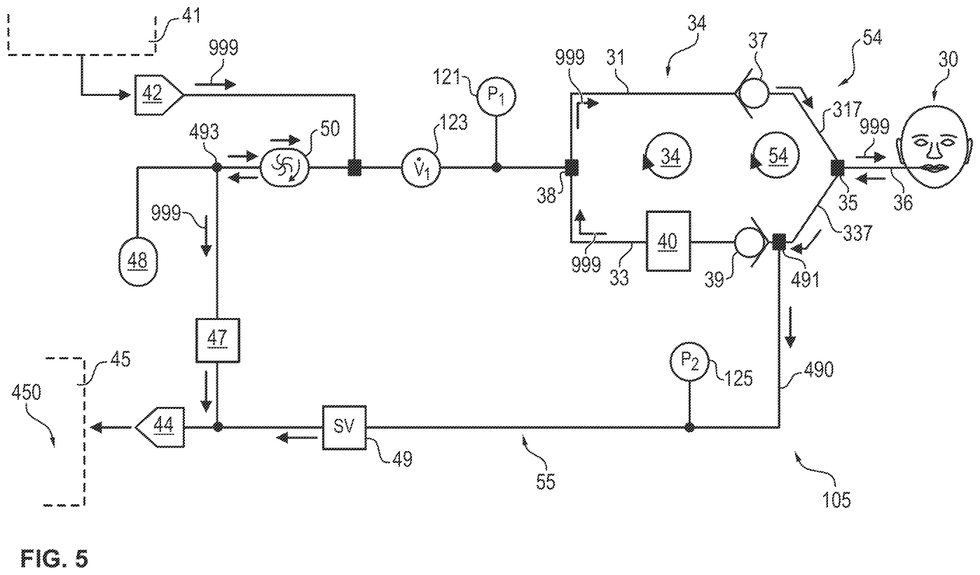

FIG. 5 shows an alternative embodiment to FIG. 2 with an assembly 105, in which the carbon dioxide absorber 40 is arranged in the expiratory path 33. Flow arrows 999 indicate the directions of gas flows in the assembly 105.

Furthermore, FIGS. 1, 2, 3, 5, 6 show another pressure sensor P2 125, which may be arranged at the expiratory path 31 or, as an alternative, also at the patient connection element (Y-piece) 35. With such an additional pressure sensor P2 125, the flush valve assembly 49 can be configured with an additional functionality as a pressure relief valve. The control unit 200 can thus bring about an opening of the flush valve SV 49 for a pressure relief in the pneumatic system 55 above a predefined pressure level P_{Limit} into the anesthetic gas scavenging system 44. A comparison of the measured signals of the additional pressure sensor P2 125 to a threshold value P_{Limit} makes it possible to bring about and to control an open state at the flush valve assembly 49 with pressure relief into the anesthetic gas scavenging system 44 in case the threshold value is exceeded.

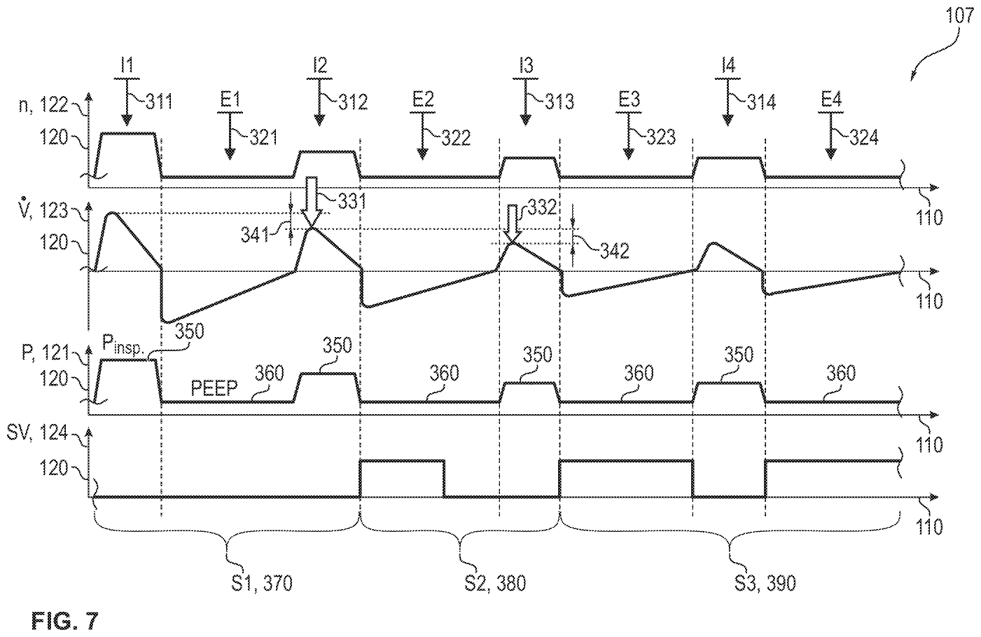

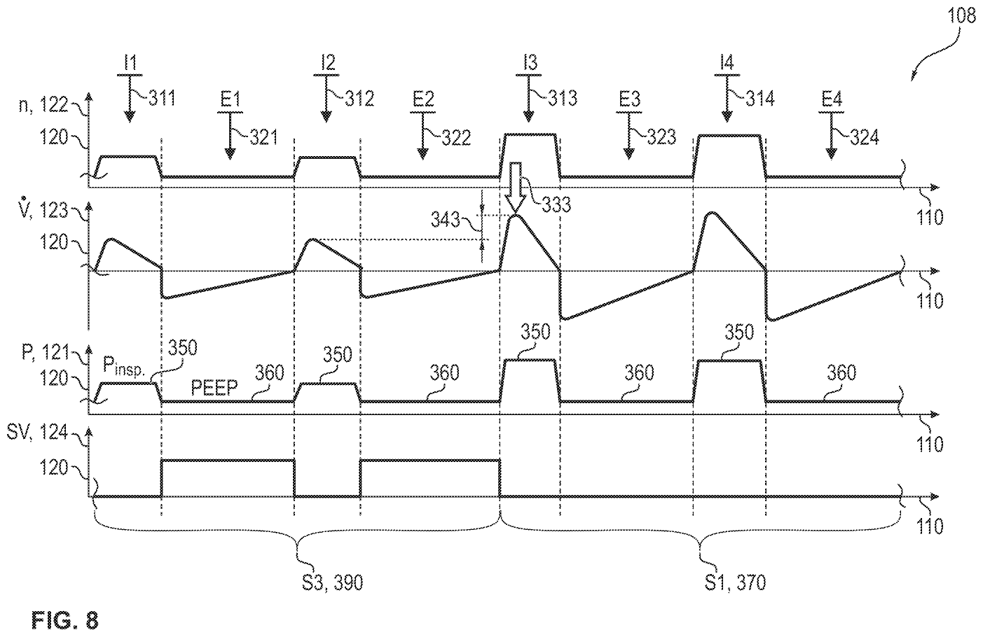

FIG. 7 and FIG. 8 show in diagrams 107, 108 in a schematic form on the x axes 110 a time curve (time course) 110 with signal curves, plotted on the y axis 120, of the ventilation pressure 121, of flow rates 123, of speed of rotation levels 122 of the radial compressor 50 and of states 124 of the flush valve assembly 49 according to the embodiment of the assembly 103 (FIG. 3). Inspiratory pressure levels 350 and a level 360 of the positive expiratory pressure (PEEP=positive end expiratory pressure=PEEP) are shown in the time curve 110 of the ventilation pressure 121. Speed of rotation levels 122 of the radial compressor 50, which belong to the respective ventilation pressures 121, 350, 360, are shown schematically in the time curve 110. Schematic curves of the volume flows, which arise from the settings or changes of settings of tidal volumes V_{T}, are shown in the time curve 110.

FIG. 7 shows in diagram 107 a variant, in which a user performs two actions with a two-step reduction of a set value of a tidal volume.

FIG. 8 shows in diagram 108 a variant, in which a user performs an action with a one-step increase in a set value of a tidal volume V_{T}.

FIGS. 7 and 8 will be described and explained below in more detail together. Phases of inhalation I1 through I4 alternate in the time curve 110 in FIGS. 7 and 8 with phases of exhalation E1 through E4, the reference numbers 311-314 are assigned to the phases of inhalation I1 through I4, and the reference numbers 321-324 are assigned to the phases of exhalation E1 through E4. Identical elements are designated by the same reference numbers in FIGS. 7 and 8.

Events 331, 332, in which the user makes a respective change in the ventilation settings (V_{T}), occur in the time curve shown in FIG. 7. The events 331, 332 in FIG. 7 represent as changes at a first time a first reduction 341 of the tidal volume V_{T} to be administered to the patient 30 (FIGS. 1 through 6) and a second reduction 342 of the tidal volume V_{T} at a second time. With the two-step reduction 341, 342 of the tidal volume V_{T} in this FIG. 7 a switching takes place from a state S1 370 of a closed anesthesia system into a state S3 390 of an open anesthesia system via a transition range of a state S2 380 of a partially open anesthesia system with a flush valve SV 49 open from time to time (FIG. 3).

An event 333, at which the user makes a change in the ventilation settings, occurs in the time curve shown in FIG. 8. The event 333 at a defined time in FIG. 8 represents as an example—and as a variation to FIG. 7—a one-step increase 343 of the tidal volume V_{T} at a time as a change. A direct switching from a state S3 390 of an open anesthesia system into a state S1 370 of a closed anesthesia system takes place with the one-step increase 343 of the tidal volume V_{T}.

FIG. 9 shows in a flow chart 109 a schematic sequence for operating an anesthesia system according to FIGS. 1 through 6 with an automatic switching between an operation as an open system and an operation as a closed system.

A start 501 is followed by a detection 502 of information, which indicates a pressure 121, 561 and flow rates 123, 562 in the pneumatic system 55 (FIGS. 1 through 8) of the assemblies 101, 101′, 102, 103, 104, 105, 106 (FIGS. 1 through 6). This detection 502 may be configured, for example, as a measured value acquisition with signal processing of measured values of the first pressure sensor P1 121 (FIGS. 1-9) 561 and of the first flow sensor V1 123 (FIGS. 1-9) 562. Identical elements in FIGS. 1 through 8 and FIG. 9 are correspondingly designated by the same reference numbers in FIGS. 1 through 9.

During the subsequent analysis 503, a determination of a current tidal volume V_{T} 565 is carried out by means of integration of the information, which indicates flow rates 562, and a comparison is subsequently carried out with predefined threshold values 563, which indicate a lower limit V_{TLimit_1} of a tidal volume and in an optional embodiment also an upper limit of a tidal volume V_{TLimit_2}. The threshold values V_{TLimit_1}, V_{TLimit_2} may also form a hysteresis, which can then be used for a subsequent case differentiation 504. The application of a hysteresis during the case differentiation 504 is advantageous in reference to the robustness of analysis 503 and case differentiation. Two principal cases 541, 551 are distinguished during the case differentiation 504 directly following the analysis 503:
the current tidal volume V_{T} 565 is lower than the predefined threshold value 563 in the first case 541 and
the current tidal volume V_{T} 565 is higher than the predefined threshold value 563 in the second case 551.

In the first case 541, i.e., in case of small tidal volumes compared to the summary volume of the internal circuit and of the external circuit, the flush valve SV 49 of the flush valve assembly 49 is opened. Gas exhaled by the patient 30 (FIGS. 1 through 6) can thus both flow back into the internal closed-circuit system 34 through the expiratory path 337, 31 (FIGS. 1 through 6) and be fed again to the patient after enrichment 41 (FIGS. 1 through 6) with additional oxygen and other gases and after processing by the carbon dioxide absorber 40 and be removed from the pneumatic system 55 (FIGS. 1 through 6) through the scavenging gas branch 490 (FIGS. 1 through 6) into the anesthetic gas scavenging system (AGSS) (FIGS. 1 through 6).

The flush valve 49 of the flush valve assembly 49 is not opened in the second case 551 and it remains in the closed state, so that gas exhaled by the patient 30 (FIGS. 1 through 6) can flow back only through the expiratory path 337, 31 (FIGS. 1 through 6) into the internal closed-circuit system 34 and can be fed again to the patient after enrichment 41 (FIGS. 1 through 6) with additional oxygen and other gases and after processing by the carbon dioxide absorber 40. With the flush valve SV 49 closed (FIGS. 1 through 6), no quantities of gas flow through the scavenging gas branch 490 from the patient 30 (FIGS. 1 through 6) to the anesthetic gas scavenging system (AGSS) (FIGS. 1 through 6).

The hysteresis can thus be configured, for example, such that when the lower threshold value 563 is undershot 541, the activation of the open state 542 of the flush valve SV 49 by the control unit 200 (FIG. 6) takes place, and, in turn, in case of exceeding 551 of the upper threshold value 563, the flush valve SV 49 is activated by the control unit 200 (FIG. 6) and the open state 552 is activated.

Following the case differentiation 504 with state control 542, 552 of the flush valve SV 49, the operation-represented by elements in the flow chart 109 with the reference numbers 505, 506, 502, 503, 504, 541, 542, 551, 552 of an anesthesia system according to FIGS. 1 through 6 with an automatic switching between an open system 542 and a closed system 552 until the end 507 of the operation—is carried out further continuously.

Optional manual possibilities for influencing the states 542, 552 are also shown in the sequence 109 according to this FIG. 9.

A possibility of manual switching is offered by a manual operating element Man.-SV 560, for example, in the embodiment as a switch, button, touch display, GUI, by which a state of change of the flush valve SV 49 between closed state 552 and open state 542 can be brought about directly.

Another possibility of switching between closed a state 552 and an open state 542 of the flush valve SV 49 can be offered by a linked operation with an additional manual operating element O₂—F. 570, which is intended for activating a so-called O₂ flush valve 572 in an anesthesia device. This additional operating element may also be configured as a manually operable operating element, e.g., as a switch, button, touch display, GUI. The switching between a closed anesthesia system and an open anesthesia system with an activation of the open state of the flush valve assembly can be triggered in this manner combined with activation of an O₂ flush state. Quantities 571 of oxygen fed through the O₂ flush valve 572 can flow during the O₂ flush state directly to the patient 30 (FIGS. 1 through 6). The inlet 571 of the O₂ flush valve 572 is usually and preferably connected for this purpose directly to the mixing unit 41 (FIGS. 1 through 6). The outlet 573 of the O₂ flush valve 572 is usually connected for this purpose in pneumatic systems 55 (FIGS. 1 through 6) directly to the inlet 493 (FIG. 6) of the radial compressor 50 (FIGS. 1 through 6). The gas exchange and the feed of oxygen to the patient 30 (FIGS. 1 through 6) can be accelerated with feed of oxygen in an O₂ flush situation by the opening 542 of the flush valve 49, which opening is combined with the O₂ flush valve 572.

The aspects and features which are described in connection with one or more of the examples and figures described in detail above may also be combined with one or more of the other examples in order to replace an identical feature of the other example or in order to additionally introduce the feature into the other example. Examples may, furthermore, be a computer program with a program code for carrying out one or more of the above processes or they may relate thereto when the computer program is executed on a computer or processor. Steps, operations or processes of different, above-described processes may be executed by programmed computers or processors. Examples may also cover program memory devices, e.g., digital data storage media, which are machine-, processor- or computer-readable and machine-executable, processor-executable or computer-executable programs of instructions. The instructions execute some or all of the steps of the above-described processes or cause them to be executed. The program storage devices may comprise or be, e.g., digital memories, magnetic storage media, for example, magnetic disks and magnetic tapes, hard drives or optically readable digital data storage media. Further examples may also cover computers, processors or control units, which are programmed for executing the steps of the above-described processes, or (field)-programmable logic arrays ((F) PLAs=(Field) Programmable Logic Arrays) or (field)-programmable gate arrays ((F) PGAs=(Field) Programmable Gate Arrays), which are programmed for executing the steps of the above-described processes. Only the principles of the disclosure are shown by the description and the drawings. All the examples mentioned here shall, furthermore, be used, in principle, expressly for illustrative purposes only in order to support the reader in understanding the principles of the disclosure and the concepts contributed by the inventor(s) to the further improvement of the technology. All the statements made here about principles, aspects and examples of the disclosure as well as concrete examples thereof comprise their equivalents. A function block designated as a “means for . . . ” executing a certain function may pertain to a circuit, which is configured for executing a defined function. Thus, a “means for something” may be implemented as a “means configured for or suitable for something,” e.g., as a component or a circuit configured for or suitable for the particular task. Functions of different elements shown in the figures, including each function block described as a “means,” “means for providing a signal,” “means for generating a signal,” etc., may be implemented in the form of dedicated hardware, e.g., of “a signal provider,” “of a signal processor unit,” of “a processor,” of “a control,” etc., as well as as hardware capable of executing software in conjunction with corresponding software. In case of provision by a processor, the functions may be provided by an individual dedicated processor, by an individual, jointly used processor or by a plurality of individual processors, some of which or all of which may be used jointly. However, the term “processor,” “control” or “regulation” is far from being limited exclusively to hardware capable of executing software, but it may comprise digital signal processor hardware (DSP hardware; DSP=Digital Signal Processor), network processor, application-specific integrated circuit (ASIC=Application Specific Integrated Circuit), field-programmable logic array (FPGA=Field Programmable Gate Array), read-only memory (ROM=Read Only Memory) for storing software, random access memory (RAM=Random Access Memory) and non-volatile storage device (storage). Other hardware, conventional and/or customer-specific, may be included as well. A block diagram may represent, for example, a general circuit diagram, which implements the principles of the disclosure. Similarly, a flow chart, a flow diagram, a state transition diagram, a pseudocode and the like may represent different processes, operations or steps, which are represented, for example, essentially in computer-readable medium, and can thus be executed by a computer or processor, regardless of whether such a computer or processor is explicitly shown. Processes disclosed in the description or in the patent claims may be implemented by a component, which has a means for executing each and every one of the respective steps of these processes. It is obvious that the disclosure of a plurality of steps, processes, operations or functions disclosed in the description or in the claims shall not be interpreted to be in a defined order, unless this is explicitly or implicitly stated otherwise, e.g., for technical reasons. Therefore, these are not limited to a defined order by the disclosure of a plurality of steps or functions, unless these steps or functions are not interchangeable for technical reasons. Further, an individual step, function, process or operation may include in some examples a plurality of partial steps, partial functions, partial processes or partial operations and/or be broken up into same. Such partial steps may be included and be part of the disclosure of this individual step, unless they are explicitly excluded. Furthermore, the following claims are included herewith in the detailed description, where each claim may stand on its own as a separate example. While each claim may stand on its own as a separate example, it should be noted that-even though a dependent claim may pertain in the claims to a defined combination with one or more other claims, other examples may also comprise a combination of the dependent claim with the subject of any other dependent or independent claim. Such combinations are proposed here explicitly, unless it is stated that a defined combination is not intended. Further, features of a claim shall also be included for any other independent claim, even if this claim is not made directly dependent on the independent claim.

While specific embodiments of the invention have been shown and described in detail to illustrate the application of the principles of the invention, it will be understood that the invention may be embodied otherwise without departing from such principles.

### LIST OF REFERENCE NUMBERS

30 Patient
31 Inspiratory path
33 Expiratory path
34 Internal closed-circuit system
35 Patient connection element (Y-piece)
36 Access, endotracheal tube
37 Nonreturn valve, inspiratory
38 Breathing system connection element (internal Y-piece)
39 Nonreturn valve, expiratory
40 Carbon dioxide absorber
41 Mixing unit for fresh gas
42 Fresh gas supply and feed
43, 43′ Fresh gas feed position
44 Anesthetic gas scavenging system (AGSS)
45 Anesthetic gas disposal
47 APL valve assembly
48 Breathing bag
49 Flush valve, flush valve assembly (purging valve)
50 Radial compressor (blower)
54 External closed-circuit system
55 Pneumatic system
101, 101′, 106 Embodiments of assemblies
102, 103, 104, 105 Embodiments of assemblies
107, 108 Diagrams with time curves
109 Flow chart, sequence
110 X axis, abscissa
120 Y axis, ordinate
121 First pressure sensor P1
122 Speed of rotation levels of the radial compressor
123 First flow sensor V1
124 States of the flush valve assembly
125 Additional pressure sensor P2
127 Additional flow sensor V2
128, 129 Filter elements
130 Anesthetic gas scavenging valve
200 Control unit
300 Data lines, signal lines
311-314 Phases of inhalation I1-I4
317 Inspiratory ventilation hose
321-324 Phases of exhalation E1-E4
331 First event
332 Second event
333 Third event
337 Expiratory exhalation hose
341 First reduction of the tidal volume
342 Second reduction of the tidal volume
343 Increase in the tidal volume
350 Inspiratory pressure level
360 Expiratory pressure level
370 Situation S1, closed anesthesia system
380 Situation S2, partially open anesthesia system
390 Situation S3, open anesthesia system
400 Control lines
424 Oxygen sensor
450 External device for anesthetic gas disposal, part of the hospital infrastructure
451 Vacuum source, vacuum
490 Scavenging gas branch (purging branch)
491 Expiratory branch
492 Branch, AGSS
493 Inlet of the radial compressor
501 Start (flow chart)
502 Detection of information
503 Analysis (flow chart)
504 Case differentiation (flow chart)
505, 506, 507 Elements in the flow chart (flow chart)
541 First case of the case differentiation (flow chart)
542 Open state of the flush valve (flow chart)
551 Second case of the case differentiation (flow chart)
552 Closed state of the flush valve (flow chart)
560 Manual operating element Man.-SV
561 Pressure information
562 Flow rate information
563 Threshold values V_{TLimit}
565 Current tidal volume V_{T}
570 Manual operating element O₂—F
571 Inlet of the O₂ flush valve
572 O₂ flush valve
573 Outlet of the O₂ flush valve
999 Flow arrows, flow directions

1. An assembly of components for a pneumatic system for an anesthesia system for providing breathing gases, with feeding and scavenging, to a patient, the assembly comprising:
a control unit;
a radial compressor as a source for providing quantities of breathing gases;
an internal closed-circuit system comprising:
a carbon dioxide absorber;
a breathing system connection element;
an inspiratory path with an inspiratory nonreturn valve; and
an expiratory path with an expiratory nonreturn valve;
a flush valve assembly;
a patient connection element;
an adjustable pressure-limiting valve (APL valve) assembly;
a breathing bag;
a mixing unit for the supply of fresh gas to the internal closed-circuit system; and
a first flow sensor,
wherein the first flow sensor is configured to detect and to provide measured signals, which indicate a flow rate flowing in the internal closed-circuit system, to the control unit,
wherein the control unit is configured to determine a current tidal volume on the basis of the measured signals, which indicate a flow rate flowing in the internal closed-circuit system, and
wherein the control unit is configured to bring about a state of change of the flush valve assembly on the basis of the current, determined tidal volume.

2. An assembly in accordance with claim 1,
wherein a first pressure sensor is arranged in the internal closed-circuit system and is configured to provide measured signals, which indicate a pressure level present in the internal closed-circuit system, to the control unit
wherein the control unit is configured to also include the measured signals, which indicate the pressure level present in the internal closed-circuit system, during the bringing about of the state of change of the flush valve assembly.

3. An assembly in accordance with claim 1, further comprising an additional pressure sensor is arranged in the pneumatic system, wherein the additional pressure sensor is configured to detect and to provide measured signals, which indicate a pressure level present in the expiratory path, to the control unit, wherein the control unit is configured to also include the measured signals, which indicate the pressure level occurring in the expiratory path, during the bringing about of the state of change of the flush valve assembly.

4. An assembly in accordance with claim 1,
wherein an additional flow sensor is arranged in the pneumatic system,
wherein the additional flow sensor is configured to detect and to provide measured signals, which indicate quantities of gas flowing from the patient, to the control unit,
wherein the control unit is configured to also include the measured signals, which indicate flow rates flowing to or from the patient, during the bringing about of the state of change of the flush valve assembly.

5. An assembly in accordance with claim 1, further comprising an oxygen sensor is arranged in the pneumatic system,
wherein the oxygen sensor is configured to detect and to provide measured signals, which indicate an oxygen concentration in the pneumatic system and/or an oxygen concentration in the internal closed-circuit system and/or an oxygen concentration of quantities of gas exhaled by the patient, to the control unit,
wherein the control unit is configured to also include the measured signals, which indicate the oxygen concentration, during the bringing about of the state of change of the flush valve assembly.

6. An assembly in accordance with claim 1, wherein the control unit is configured to also take into account measured signals of the first pressure sensor and/or of the first flow sensor, and/or of the additional pressure sensor and/or of the additional flow sensor and/or of the oxygen sensor during the control of the operation with bringing about of changes of state of the flush valve assembly, of the APL valve assembly, of the radial compressor, and of the mixing unit for fresh gas.

7. An assembly in accordance with claim 1, wherein the control unit is configured to carry out an activation of the flush valve assembly into an open state along with an activation of an additional valve.

8. An assembly in accordance with claim 1, wherein the flush valve assembly is configured with an additional functionality as a pressure relief valve.

9. A process for operating an anesthesia device or ventilator with a pneumatic system, comprising: a control unit; a radial compressor as a source for providing quantities of breathing gases; an internal closed-circuit system comprising: a carbon dioxide absorber; a breathing system connection element; an inspiratory path with an inspiratory nonreturn valve; and an expiratory path with an expiratory nonreturn valve; a flush valve assembly; a patient connection element; an adjustable pressure-limiting valve assembly; a breathing bag; and a mixing unit for the supply of fresh gas to the internal closed-circuit system, the process comprising the steps of:
acquiring a measured signal with a detection of measured signals of a first pressure sensor and of a first flow sensor;
with a measured value analysis, determining an operating state of the anesthesia system on the basis of the measured signals; and
adapting an operation of the anesthesia system with control of the flush valve assembly as a function of the determined operating state in a sequence of steps, and
wherein the determination is carried out to determine whether an operating state is present, in which certain partial quantities or quantities of carbon dioxide-containing and oxygen-depleted exhaled gases, which are exhaled by the patient, flow back to the patient.

10. A process for operating an anesthesia system in accordance with claim 9,
wherein the measured signal analysis takes place such that the current inspiratory tidal volume is calculated during the phase of inhalation on the basis of the measured signals of the first flow sensor and the current tidal volume is compared to a threshold value, and
wherein the adaptation of the operation of the anesthesia system is carried out such that in case of an undershooting of the threshold value by the current tidal volume, an activation of the flush valve assembly into an open state takes place.

11. A process for operating an anesthesia system in accordance with claim 10, wherein the activation of the flush valve assembly into the open state does not take place during each phase of breathing-, but only from time to time or proportionally.

12. A process for operating an anesthesia system in accordance with claim 9,
wherein the activation of the flush valve assembly takes place with transition from a closed anesthesia system to an open anesthesia system over a first transition range of a defined volume range of tidal volumes and/or
wherein the deactivation of the flush valve assembly takes place with transition from a closed anesthesia system to an open anesthesia system over a second transition range of a defined volume range of tidal volumes.

13. A process for operating an anesthesia system in accordance with claim 12, wherein an alternation between a closed anesthesia system and an open anesthesia system is controlled
on the basis of information concerning an expiratory volume and/or
on the basis of information pertaining to an oxygen concentration in the breathing gas.

14. A process for operating an anesthesia system in accordance with claim 12, wherein an alternation or a switching between a closed anesthesia system and an open anesthesia system
is triggered with an activation of the open state of the flush valve assembly combined with an activation of an O₂ flush state and
with an activation and/or deactivation of the open state of the flush valve assembly by a manual input.

15. A process in accordance with claim 9, wherein a computer program with a program code provided with a non transitory computer readable media for carrying out at least some of the processes steps when the program code is executed on a computer, on a processor or on a programmable hardware component.

16. An assembly of components for a pneumatic system for an anesthesia system for providing breathing gases the assembly comprising:
an internal closed-circuit system comprising:
a carbon dioxide absorber;
a breathing system connection element;
an inspiratory path with an inspiratory nonreturn valve; and
an expiratory path with an expiratory nonreturn valve;
a patient connection element connected to the internal closed-circuit system;
a gas reservoir;
a radial compressor as a source for providing quantities of breathing gases from the reservoir to the internal closed-circuit system at the breathing system connection element; and
a filter between the radial compressor and the breathing system connection element.

17. An assembly of components for a pneumatic system for an anesthesia system for providing breathing gases the assembly comprising:
an internal closed-circuit system comprising:
a carbon dioxide absorber;
a breathing system connection element;
an inspiratory path with an inspiratory nonreturn valve; and
an expiratory path with an expiratory nonreturn valve;
a patient connection element connected to the internal closed-circuit system;
a gas reservoir;
a radial compressor as a source for providing quantities of breathing gases from the reservoir to the internal closed-circuit system at the breathing system connection element;
a flush branch leading from the internal closed-circuit system;
a flush valve in the flush branch; and
a filter in the flush branch.
